(19) United States
(12) Patent Application Publication
(10) Pub. No.: US 2008/0206854 A1
(43) Pub. Date: Aug. 28, 2008

(54) Reactors (medical devices) in which chemical, biochemical, biological reactions - activations take place and their final products are used

(76) Inventors: Eduardos Spiridon Tsakas, Athens (GR); Spiridon Tsakas, Athens (GR)

Correspondence Address: MATHEWS, SHEPHERD, MCKAY, & BRUNEAU, P.A., 29 THANET ROAD, SUITE 201, PRINCETON, NJ, 08540, US

(21) Appl. No.: 12/072,249

(22) Filed: Feb. 25, 2008

(30) Foreign Application Priority Data
Feb. 23, 2007 (GR) 20070100115

(51) Int. Cl. C12M 1/00; B01J 19/00

(52) U.S. Cl. 435/287.2; 435/289.1; 422/129

(57) ABSTRACT

Reactors (medical devices), which contains one or more independent of each other solutions and has two or more openings with corresponding indented hollow caps and/or pump and/or needle on the other opening as delivery system. The caps contain within them sensitive or incompatible enzymes, chemical, biological, or synthetic substances in powder or tablet form. Indicative of these caps is the fact that when impressed or twisted the protective seal within is broken, releasing their content into the solution or solutions contained within the vial in the order where the desired reactions will take place and the final products will be used by the user for pharmaceutical, cosmetic, nutritional, diagnostic, and other purposes.

### DESCRIPTION OF THE INVENTION

1. Technical Field of the Invention

The reactor contains two or more hollow corks-lids within which the interacting chemicals or biological substances (enzymes, activators, inhibitors, reactants etc.) are kept separately in their natural dry or liquid form and one or more buffer solutions where the reaction (s) will take place giving us the final substances (final products) for medical, pharmaceutical, nutritional, diagnostic, cosmetic and other applications to be used freshly prepared. In addition one of the corks can be a pump or injection needle when the final solution—product is going to be sprayed or injected.

2. Prior Art

It is known that enzymes can not be preserved within solutions or in the same mixture, while their encapsulation into liposomes is unsuccessful due to their large molecular size. Furthermore, the simultaneous use of two enzymes may not be possible, or might be very difficult due to their counterreactions or to the fact that their reactions require different conditions.

One such example are peptidases which can not coexist with lipase enzymes or with other enzymes in general because the first will use the latter as a substrate (thereby destroying them) while their reactions usually require much different pH levels in order to occur, from pH 3 to pH 5 for peptidases and from pH 7 to pH 9 which lipases require.

Additionally, in many cases the reaction of the mixture of different enzymes and/or reactants and/or substances is desired to take place shortly prior to application/use, meaning a freshly active created mixture to be used, when for example one of the factors or the final product has a short life span.

There is also the case of two substances, for example one oxidizing and the other reducing, which cannot coexist together for long periods of time in the same mixture or solution.

Up until now, there have been no reactors/vials with double or multiple indented hollow caps (with or without a pump or needle as a delivery system on the other side), and also possessing one or more solutions for mixture in which the desired reactions will be taking place independently of one another or in a series within the same solution with the purpose of being produced by the user and made active right after their mixture and just prior to their application.

The present invention provides for the first time the solution to this specific problem(s), using the simplest, useful, effective, and cheapest productive method possible as of today.

Thus, this invention provides a solution, which exists or arises in a certain procedure in many areas of the sciences (biological or physical) and their applications when enzymes and/or reactants and/or substances cannot coexist together due to their interactions in the same dry phases (powders) or in the same liquid phases (solutions).

### SUMMARIZED PRESENTATION OF THE INVENTION

The present invention describes containers in the form of vials which contain one or more solutions and have two or more indented necks/hollow caps and a pump or needle as the delivery system on the other side, for separate placement/preservation in either dry (powder) or liquid (solution) phase of incompatible enzymes, reactants, and substances for use of the above after their mixture in solutions for pharmaceutical, cosmetic, diagnostic and other purposes.

In such cases where two substances for example one oxidizing and the other reducing which can not co-exist in the same mixture or solution, this invention provides a solution to this problem.

This description illustrates demonstrative examples of possible applications for this invention, which are only a limited sample of the wide range of possible applications for which this invention can be used.

The aforementioned invention describes a reactor/vial, which contains one or more independent of each other solutions and have two or more openings, which are covered by indented hollow caps, pump or needle placed at the other opening. In the compartment of the indented hollow caps exist sensitive or incompatible enzymes, chemical, biological, or synthetic substances in powder or tablet form. A characteristic feature of these caps is that when they are impressed or twisted, they break the existing seal within and release their reactive contents within the solution or solutions which are contained in the vial in the sequence that the user ordered with the purpose of immediate use by the client of the finalized product for pharmaceutical, cosmetic, nutritional, diagnostic or other purposes.

A further advantageous application of this invention is that the aforementioned vial designated by the fact that it contains two or more caps in which different enzymes are preserved which can not be combined based on their reactions (for example peptidases and lipases), their reaction pH levels, the use of their activators, or their coenzymes.

By means of convenience, the above-proposed vials have applications in cosmetics and primarily in the removal of acne symptoms, the deep skin cleansing with use of skin renewing factors, its renewal, and cosmetic treatment for the breakdown/removal of lipids from lipomas and cellulite without the need for surgery.

Also, the substances stored in vials which this invention describes are characterized by the fact that they are viewed as substrates when they are in a liquid phase or in the hollow cap where they are in a dry form as human epidermal cell development agents in cell cultures and used for the renewal of epidermal tissues and the macromolecules which bind cells together such as collagen and elastin, for the reduction of symptoms of ageing such as wrinkles, looseness, and undesired colorations.

Additionally the indented vials may also contain in their caps or solutions epidermal agents or factors such as EGF (Epidermal Growth Factors) and SRF (Skin Remodeling Factors) with the purpose of renewing/reconstructing the skin, and accelerated healing of wounds, scars, etc.

By means of convenience, within the vials of this invention necessary compounds are also contained which are incompatible with one another, such as molecular oxygen (02), ascorbic acid, iron ions (Fe++), ketoglutaric, and/or pyruvic acid and their salts with the purpose of synthesizing new collagen and elastin fibers by the epidermal cells.

When there is molecular oxygen produced within the solution of the vial, it can be used via a pump placed on its other side.

The vial can also contain, in combination with another vial, or in and of itself the enzyme catalase in its hollow cap and hydrogen peroxide in its solution thus making it possible for the user to produce a solution containing molecular oxygen, for immediate application in the oxygenation, sterilization of the surface skin, or oxygenation of cavities for removal of odor such as in the areas of arm pits, the mouth, the groin area, and others due to its antibacterial action.

In addition, this vial is also capable of containing ketonoacids and specifically pyruvic acid or its salts in the solution in any concentration from 0-10%.

Furthermore, in the caps of the reactors/vials it is possible to contain antibodies A and B or others (based on antibodies interactions) with the purpose of instant identification of whichever blood type and other hematological characteristics of antibodies or cell compatibility, as well as the identification of the presence of contaminating agents such as the HIV virus or hepatitis in the blood, or drug uses for immediate diagnosis by the client(s).

### DETAILED DESCRIPTION OF THE INVENTION

The aforementioned vial can also be used in a cylindrical shape reactor with two indented hollow caps at each end. In this way, the user will be able to impress one cap at a time, in this case one different enzyme or reactant, which is in the cap. Thus, the user is able to use each enzyme at a time, impressing the cap on the other side when he/she wishes to use the alternate enzyme or reactant present in the other cap. Consequently, the user performs two different chemical or enzymatic reactions in a row in the same solution and measuring the final products concentration, activity etc.

With the integration of the first enzyme from the first cap, this is achieved by impressing the first cap, the first enzyme which is contained within the cap will be released into the solution of the cylinder, and modify the initial substrate which is contained in the solution, thereby creating a new substrate. This is followed by the integration of the second enzyme from the second cap, which will further modify the newly formed substrate, thus creating the final substrate of the product, which will be used by the client.

In the possible application of this cylinder for diagnostic purposes, the finalized substrate will be the one, which will provide the client with the final identification/diagnosis, which he/she desires.

There are also advantages in the use of a double vial with two indented hollow caps and two separate solutions. This double vial can either take the form of one vial divided into two compartments within it. This allows for the separate storage of two solutions and have two different caps each next to its solution.

One application of this invention for diagnostic purposes is when in the first cap antibodies of blood type A are present and in the second cap antibodies of blood type B are present has an adjacent entry or temporarily removing the cap for the insertion of a small quantity of blood into its solution. Then we insert small quantities of blood into the separate solutions, close the entryways and continue by releasing the antibodies of A into the first vial, and the antibodies of B into the second vial. Using this technique we will be able to identify the blood type of the donor, based on antibodies reactions, quickly and easily, even in the case of an injured patient being transported to the hospital by ambulance for example.

Another useful application of this invention, is the use of this double vial with two indented hollow caps in the case where two users need simultaneous testing for the presence of antibodies of the proteinic envelop of the HIV virus and/or hepatitis in two caps of the respective AIDS and/or hepatitis diseases shortly prior to their consummation or for one user to test himself for the existence of both pathogens antibodies.

Another suggested possible application of this patent for the aforementioned acne problems or for enzymatic skin cleansing is the presence of two vials connected at their bases in a cylindrical shape again, in which each vial has its own hollow cap and solution. The first vial could have a buffer with an alkaline pH of 7-8 and other factors necessary for lipase action while in its cap may contain certain lipases for the breakdown of sebum in the sebaceous glands, which clog the pores resulting in acne symptoms. With the release of the lipase powder into the solution of the first vial, an active enzymatic solution is created for cleansing sebum and oily skin, with simultaneous unclogging of the sebaceous glands, breaking down the sebum which clogs them. The second vial in this example could have a buffer with an acidic pH of 3-5 and could contain specially selected peptidase(s) and/or keratinase(s) which will break down the cell walls and macromolecules of the epidermal cells and primarily those which connect one another such as involucrine, proteoglycans, etc.

By applying the first active solution, and following that applying the second active solution to the skin area, we have managed to compose an active enzymatic solution for facial cleansing and general skin cleansing from dead cells fat and keratine molecules which exist on the skin, which also accomplishes the unclogging of clogged sebaceous glands by removing dead cells and complexes formed by the dead cells thereby opening them and drastically reducing the acne symptoms, creating with their opening aerobic conditions within the gland.

The importance of this invention is made even more apparent by the fact that it is extremely difficult and costly to preserve lipases and peptidases together in the same single cap of a vial and consequently together in a single solution for two main reasons: one is the fact that their buffer pH levels are different, and also because the peptidases will break the lipases down to peptides making them inactive. The applications of this reactor provide a solution to all the above mentioned problems, as well as others of a similar sort.

There is an additional beneficial application of the invention which is that the double vial could also be made to serve pharmaceutical purposes as well. In such a case we could have a micro injection of the second aforementioned active solution (i.e. peptidases), which was produced according to the application of the invention, in a small or large lipoma therefore the injected peptidases will be able to break down the cell walls of the lipoma's fat cells. This, followed by an injection of the primary aforementioned lipases will achieve the breakdown, liquidize and release of the cleared out fat from the lipomas which can then be sucked out very easily for example with a suction needle used on the lipoma. In this way, the skin area affected by the lipoma can return to its prior healthy and normal appearance.

The above process can be also used in carcinomas or melanomas by using in the mixture specific factors such as TNF (tumor necrosis factors).

Additionally, in cases where there is an area of scattered lipomas or a singular spread deposit of hypodermal fat such as in the case of cellulite in the thighs or other areas, instead of the use of a single microinjection for the release of the enzymatic solutions as above, it is possible to conduct a series of micro injections simultaneously via an existing supplier.

In this way we manage to avoid surgeries, liposuctions, possible contaminations, or side effects and pain with a simple, compatible, and cost effective procedure with multiple benefits made available to the final consumer.

Finally, the above described device is applicable also when the first vial provides the reactive substrate to be used and the other one (on the other side) to contain, provide the necessary reactants (freshly produced) antidotes, when an over reaction will be the outcome from the prior used reactive substrate.

In summary, the advantages of the invention in its different applications is that it gives the opportunity to: 1) preserve sensitive powders/substances separately for long periods of time 2) solutions made active by the user easily moments prior to its application 3) produce smart cosmetic products because with its application it is possible to achieve many specialized and selected actions to take place in small areas, such as the opening of each clogged sebaceous gland in skin with acne symptoms and 4) help prevent future establishment of acne symptoms.

There are also beneficial applications of this invention in the cosmetic sector in the case as reported above where a client has an area of skin (face for example) with open sebaceous glands and without any acne symptoms, lipomas, etc. anymore and has new cells at its surface. These new cells need vital nutrients and ingredients for their normal development, functioning, reproduction and renewal. In some cases, certain of these necessary ingredients are no longer being provided to the cells from the organism due to ageing and/or there is an evident presence of symptoms of ageing such as wrinkles, looseness, hypoxia or discolorations.

Currently, in laboratories, epidermal human and animal cells are being grown in cultures. These cells multiply on their own in vitro in the cell cultures and also produce extracellular connective macromolecules (i.e. collagen, elastin). These macromolecules are mainly responsible for the tight/youthful appearance of the skin, and are lost due to ageing, resulting in looseness and wrinkles.

These cell cultures media are being used in laboratories where beyond the use of human and animal cells, there is also work being done on the substances which these cells produce such as collagen, EGF (Epidermal Growth Factor), etc.

However, the substances' materials which have been given to the cell cultures via their nutritional media have not been used until today in cosmetic products due to certain difficulties. The present invention provides a solution for these cells external nutritional feeding as well and makes their use in cosmetic products possible furthermore.

A possible fruitful application of this invention is in the aforementioned cell cultures, by using a third double cork vial or into the other two vials with which the skin cleansing was done, there is such necessary ingredients (cell culture's media) from which the dry form are placed into the caps, and the rest of the ingredients in liquid phase within the solution of the vials, there are integrated creating the finalized cell's nutritional solution which can be applied directly to the freshly cleaned facial skin.

Thus, the user's approach to the above skin problems will be complete, and he/she will achieve excellent results with renewal, and remodeling of their skin.

The proposed fruitful application of this invention can also be used for products to treat the healing of wounds or scars with the addition of factors such as:
1) SRF—Skin Remodeling Factors (micropeptides)
2) EGF—Epidermal Growth Factors (micropeptides)

In this way we achieve the complete renewal of the surface skin, even in cases of wounds, small scars or burns, coming closer to a more youthful, pre scarring appearance without major alterations or modifications such as those, which take place after procedures such as plastic surgery.

A further application of this invention which is also being claimed is the use of substrates or solutions of epidermal cell development for laboratory or industrial to be used now in cosmetics and the production of cosmetics with some or total incorporation of these elements.

For the realization of the application regarding cell cultures it is possible to use within the reactors/vials for instance the development solutions which are referenced in the book “Biochemical Engineering and Biotechnology Handbook” Second Edition (Stockton Press) by Bernard Atkinson and Ferda Mavituna, in the chapter “Animal Cell Culture” (pgs. 447-519).

Eagle's substrate has been successfully tested in preliminary experiments of ours in cosmetics for cosmetic applications.

The use of pyruvic acid and/or its salts also comprises a suggested application of the invention and is claimed within it since it can become an ingredient of one vial, in the case that it is in acid form it will be placed in the solution and in the case that it is in salt form it will be placed within the compartment of the hollow cap.

Pyruvic acid and its salts are of crucial importance because it has been found to be capable of:

a) converting to ethanol (from anaerobic bacteria)

b) converting to lactic acid in categories of microorganisms as well as in human cells when there is lack of oxygen.

c) converting to the pivotal acetylcoenzyme A in the mitochondria where it finally takes part in the transfer of electrons in oxygen and metabolism in general.

Thus, not only does the integration of pyruvic acid reduce the presence of acne symptoms, but it can also be converted to lactic acid in cells where the surface skin have limited amounts of oxygen available to them (as occurs in ageing) and have production of lactic acid by the cells and finally enhances the oxydoreducing activity of epidermal cells with the creation by them of the acetylcoenzyme A.

Another possible application of this invention is the following:

It is known that for the synthesis of collagen and elastin by the surface skin cells, amino acids, molecular oxygen, ascorbic acid, ketoglutaric, Ferrum ions and possibly pyruvic acid are required. Thus to expedite the production of collagen and elastin by the skin when it has lost this ability even more, these ingredients especially ascorbic acid and molecular oxygen need to be preserved separately and mixture produced shortly prior to their application. Here is where the invention steps in with the solution to this problem as well.

A double reactor/vial with two caps and two solutions is used; the first vial contains within its solution a quantity of hydrogen peroxide which has a low pH level and/or acetanilide as a stabilizer and the cap (dry phase) contains the catalase enzyme. With the release of the catalase and its integration into the solution, molecular oxygen will be enzymatically produced which due to the increased pressure (the cap is not removed but rather left in place) is dissolved into the solution. This molecular oxygen can be applied to the skin as a sterilizer or a source of molecular oxygen while after its use the product of the second vial can be used which contains all the supplemental ingredients necessary (like ascorbic acid). With the use of this invention, the application of these incompatible factors is made possibly where the skin needs them.

The vial containing the molecular oxygen can also be used on its own if at one of its ends has a pump. This way each time the user can apply a given quantity of molecular oxygen by pressing down on the pump without losing the rest of the dissolved molecular oxygen contained within the vial's solution.

In these descriptions are contained also some of the possible applications for this invention, while it is apparent that this invention also covers the use of thermoresistant enzymes, isolated from thermophile bacteria where the enzymes after heat treatment remain quite active. The high temperatures, even that of boiling point, for time periods of up to tens of minutes do not neutralize or deactivate them and thus we achieve a greater life span for the produced enzymatic products cosmetics.

It goes without saying that the shape and material of the make of the vial or its caps may differ. Also the description of the vial as a cylindrically shaped objected in the present invention is descriptive and consequently the construction of this vial is possible with a vial of any shape as long as the above contains two or more indented hollow caps or one cap and a pump or an injection on the other side.

1. Reactors (medical devices), which comprise one or more solutions and have two or more openings (necks) with corresponding indented hollow caps for its openings. The caps contain within them sensitive or incompatible enzymes, chemical, biological, or synthetic substances in powder or tablet form. Indicative of these caps is the fact that when impressed or twisted the protective seal (diaphragm) within is broken, releasing their content into the solution or solutions contained within the vial. Additionally, one of the corks on the other side can be a pump or injection needle when the final solution-product is going to be sprayed or injected. The reactions (chemicals, enzymatic etc.) take place within the solution(s) and their end-products are used in the order the user chooses or is directed for pharmaceutical, cosmetic, nutritional, diagnostic or other purposes.

2. Reactor according to claim 1 characterized by the presence of two or more caps in which enzymes are preserved separately since they can not be combined due to their interactions (for example peptidases and lipases) pH levels, the use of their activators, coenzymes, etc., so that the desired enzymatic reactions is utilized.

3. Reactor according to claim 1, characterized by its application in cosmetics, and primarily for the treatment of acne, or deep skin cleansing with the use of factors for skin renewal/remodeling and in the breakdown/removal of fat from lipoma and cellulite without surgery.

4. Device according to claim 1 characterized by the fact that they are viewed as substrates when they are in a liquid phase or in the hollow cap where they are in a dry form and after mixing form the final substrates used as human epidermal cell development agents for the renewal/remodeling of epidermal cells and the macromolecules which hold them together such as collagen and elastin. By doing so, the symptoms of ageing such as wrinkles, looseness, and undesired colorations are reduced.

5. Device according to claim 1 characterized by the fact that its indented hollow caps may also contain within their caps or solutions epidermal agents such as EGF (Epidermal Growth Factors) and SRF (Skin Renewing Factors) with the purpose of remodeling the skin, and accelerated healing of wounds, scars, etc.

6. Reactor according to claim 1 characterized by the fact that within the vials of this invention fundamental agents/ingredients are also contained which are incompatible to each other, such as molecular oxygen (02), ascorbic acid, Ferrum ions (Fe++), ketoglutaric, and pyruvic acid and their salts with the purpose of the synthesis of new collagen and elastin fibers by the epidermal cells.

7. Device according to claim 1 characterized by having a pump or injecting needle on the other end of the vial so the final product to be used in a spray (like dissolved molecular oxygen) or injected solution (lipases, peptidases).

8. Reactor according to claim 1 characterized by the fact that in the caps of the vials it is possible to contain antibodies A and B or other with the purpose of instant identification of whichever blood type and other hematological characteristics of antibodies or cell compatibility, as well as the identification of the presence of contaminating agents such as the HIV virus or hepatitis in the blood, for immediate diagnosis by the client(s) and/or doctors.

9. Reactor according to claim 6 characterized by the fact that the vial can also contain, in combination with another vial, or in and of itself the enzyme catalase in its hollow cap or hydrogen peroxide in its solution thus making it possible for the user to produce a solution containing molecular oxygen, for immediate application in the oxygenation, sterilization of the surface skin, or oxygenation of cavities for removal of odor such as in the areas of arm pits, the mouth, the groin area, and other due to the breakdown of the external cell membrane of the bacteria present there.

10. Device according to claim 6 characterized by the fact that this vial is also capable of containing ketonoacids and specifically pyruvic acid or its salts in the solution in any concentration from 0-10%.
